(19) United States
(12) Patent Application Publication
(10) Pub. No.: US 2018/0280707 A1
(43) Pub. Date: Oct. 4, 2018

(54) PULSE WIDTH MODULATED AMPLITUDE MODULATION

(71) Applicant: QUALCOMM Incorporated, San Diego, CA (US)

(72) Inventor: Stephen Jay Shellhammer, Ramona, CA (US)

(21) Appl. No.: 15/687,148

(22) Filed: Aug. 25, 2017

Related U.S. Application Data
(60) Provisional application No. 62/480,945, filed on Apr. 3, 2017.

(51) Int. Cl. A61N 1/372; A61N 1/378; A61B 5/0476; A61B 5/00

(52) U.S. Cl. CPC A61N 1/37223; A61N 1/37205; A61B 5/686; A61B 5/0476; A61N 1/3787

(57) ABSTRACT

Techniques provided herein are directed toward providing a robust pulse width modulated (PWM) amplitude modulation scheme that enables medical implants with highly inaccurate LOs to reliably receive downlink communications from an interrogator device by using pulse width to encode data. Synchronization and data fields can comprise pulse pairs, distinguishable by medical implants from a ratio of the duration of a long pulse to the duration of a short pulse within the pulse pair. Thus, synchronization can be easily and robustly obtained

### RELATED APPLICATIONS

This application claims the benefit of U.S. Provisional Application No. 62/480,945, filed Apr. 3, 2017, entitled “PULSE WIDTH MODULATED AMPLITUDE MODULATION”, of which is assigned to the assignee hereof, and incorporated herein in its entirety by reference.

### BACKGROUND

A wireless medical implant system for a patient can comprise an interrogator device, typically in, on, or in proximity to the patient, and a plurality of electronic medical implants that can take biological measurements of a body part (e.g., biological tissue) and communicate them to the interrogator device. The interrogator device can then communicate this information to other devices, such as a mobile phone, tablet, or medical device of the patient or patient's healthcare provider. The interrogator device can also communicate with the medical implants to cause them to stimulate the body part.

However, the medical implants may need to operate on very low power consumption. This can provide severe power constraints on the design of the local oscillator used for wireless communication. Thus, to preserve power in a medical implant system, wireless communication may need to tolerate relatively large inaccuracies in the carrier frequency.

### SUMMARY

Techniques provided herein are directed toward providing a robust pulse width modulated (PWM) amplitude modulation scheme that enables medical implants with highly inaccurate LOs to reliably receive downlink communications from an interrogator device by using pulse width to encode data. Synchronization and data fields can comprise pulse pairs, distinguishable by medical implants from a ratio of the duration of a long pulse to the duration of a short pulse within the pulse pair. Thus, synchronization can be easily and robustly obtained.

An example method of decoding wireless information at a receiving device, according to the description, comprises receiving, at the receiving device, a communication frame transmitted via an amplitude-modulated radio frequency (RF) signal and comprising a pair of successive pulses, where the pair of successive pulses comprises a first pulse and a second pulse, and an amplitude of the first pulse is different than an amplitude of the second pulse. The method further comprises determining a ratio of a duration of the first pulse to a duration of the second pulse, comparing the ratio with a threshold, and in response to comparing the ratio with the threshold, synchronizing a timing of the receiving device to track one or more data fields of the communication frame.

The method may comprise one or more the following features. The method may further comprise using a first counter to determine the duration of the first pulse, and using a second counter to determine the duration of the second pulse. The method may further comprise using a comparator to distinguish the amplitude of the first pulse from the amplitude of the second pulse. The method may further comprise receiving, in a data field of the communication frame, a second pair of successive pulses, where the second pair of successive pulses comprises a third pulse and a fourth pulse, and the amplitude of the third pulse is different than the amplitude of the fourth pulse, and further comparing a duration of the third pulse with a duration of the fourth pulse, and determining a digital bit of information encoded by the third pulse and the fourth pulse based on the comparison of the duration of the third pulse with a duration of the fourth pulse. The method may further comprise determining, from the communication frame, a data field corresponding to the receiving device by counting a number of bits in the communication frame. The method may further comprise powering the receiving device, at least in part, using energy from the amplitude-modulated RF signal. The receiving device may comprise a medical implant. The medical implant may be implanted in a patient's brain.

An example method of encoding wireless information at a transmitting device, according to the description, comprises generating, at a transmitting device, a communication frame having a synchronization field where the synchronization field comprises a pair of successive pulses having a first pulse and a second pulse, and a ratio of a duration of the first pulse to a duration of the second pulse exceeds a threshold. The method further comprises sending, with the transmitting device, the communication frame via an amplitude-modulated radio frequency (RF) signal such that an amplitude of the first pulse is different than an amplitude of the second pulse.

The method may comprise one or more the following features. The method may comprise encoding, in the communication frame, a digital bit of information in a third pulse and a fourth pulse, wherein a value of the digital bit is based on a ratio of a duration of the third pulse compared to a duration of the fourth pulse, and wherein the ratio of the duration of the third pulse compared to a duration of the fourth pulse does not exceed the threshold. The transmitting device may comprise an interrogator device configured to communicate with a medical implant implanted in a patient's brain.

An example medical device, according to the description, comprises a wireless communication interface configured to receive a communication frame transmitted via an amplitude-modulated radio frequency (RF) signal comprising a pair of successive pulses where the pair of successive pulses comprises a first pulse and a second pulse, and an amplitude of the first pulse is different than an amplitude of the second pulse. The medical device further comprises a memory and a processing unit communicatively coupled with the wireless communication interface and the memory and configured to determine a ratio of a duration of the first pulse to a duration of the second pulse, compare the ratio with a threshold, and synchronize a timing of the medical device, based on the comparison of the ratio with the threshold, to track one or more data fields of the communication frame.

The medical device may further comprise one or more of the following features. The medical device may comprise a first counter configured to determine the duration of the first pulse, and a second counter configured to determine the duration of the second pulse. The first counter and the second counter may compose part of the wireless communication interface or the processing unit. The medical device may further comprise a comparator configured to distinguish the amplitude of the first pulse from the amplitude of the second pulse. The comparator may compose part of the wireless communication interface or the processing unit. The wireless communication interface may be further configured to receive, in a data field of the communication frame, a second pair of successive pulses, where the second pair of successive pulses comprises a third pulse and a fourth pulse and the amplitude of the third pulse is different than the amplitude of the fourth pulse, and the processing unit may be further configured to compare a duration of the third pulse with a duration of the fourth pulse, and determine a digital bit of information encoded by the third pulse and the fourth pulse based on the comparison of the duration of the third pulse with a duration of the fourth pulse. The wireless communication interface or the processing unit may be further configured to determine, from the communication frame, a data field corresponding to the medical device by counting a number of bits in the communication frame. The wireless communication interface or the processing unit may be configured to count the number of bits in the communication frame using a counter. The medical device may be further configured to be powered, at least in part, using energy from the amplitude-modulated RF signal. The medical device may comprise a medical implant. The medical implant may be configured to be implanted in a patient's brain.

An example interrogator device, according to the description, comprises a memory and a processing unit communicatively coupled with the memory. The processing unit is configured to generate a communication frame having a synchronization field where the synchronization field comprises a pair of successive pulses having a first pulse and a second pulse, and a ratio of a duration of the first pulse to a duration of the second pulse exceeds a threshold. The interrogator device further comprises a wireless communication interface communicatively coupled with the processing unit and configured to send the communication frame via an amplitude-modulated radio frequency (RF) signal such that an amplitude of the first pulse is different than an amplitude of the second pulse.

Interrogator device may comprise one or more the following features the processing unit or wireless communication interface maybe further configured to encode, in the communication frame, a digital bit of information in a third pulse and a fourth pulse, wherein a value of the digital bit is based on a ratio of a duration of the third pulse compared to a duration of the fourth pulse, and wherein the ratio of the duration of the third pulse compared to a duration of the fourth pulse does not exceed the threshold. The interrogator device may be configured to communicate with a medical implant implanted in a patient's brain.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is a simplified cross-sectional diagram illustrating an embodiment of a wireless medical implant system.

FIG. 2 is an illustration of how data can be encoded as pulse pairs in the PWM amplitude modulation scheme, according to embodiments.

FIG. 3 is a schematic diagram of a circuit that may be used by a medical implant to decode a signal encoded in the manner illustrated in FIG. 2.

FIG. 4 is an example binary output of the slicer illustrated in FIG. 3.

FIG. 5 is a diagram of a communication frame received in the downlink signal that may be used for synchronization of the medical implants, according to embodiments.

FIG. 6 is a flow diagram of an example method of decoding wireless communication at a receiving device, according to another embodiment.

FIG. 7 is a flow diagram of an example method of encoding wireless information at a transmitting device, according to an embodiment.

FIG. 8 is a simplified block diagram of an interrogator device, according to an embodiment.

FIG. 9 is a simplified block diagram of a medical implant, according to an embodiment.

Elements, stages, steps, and actions in the figures with the same reference label in different drawings may correspond to one another (e.g., may be similar or identical to one another). Further, some elements in the various drawings are labelled using a numeric prefix followed by a numeric suffix (where the numeric prefix and the numeric suffix are separated by a hyphen). Elements with the same numeric prefix but different suffices may be different instances of the same type of element. The numeric prefix without any suffix is used herein to reference any element with this numeric prefix.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

It will be understood by a person of ordinary skill in the art that, although the embodiments provided herein are directed toward medical applications, the techniques described herein may be utilized in other applications involving digital communication. Additionally, embodiments provided herein describe the use of “medical implants,” although such implants may be utilized to gather data and/or stimulate a body part without necessarily performing a medical function. A person of ordinary skill in the art will recognize many variations.

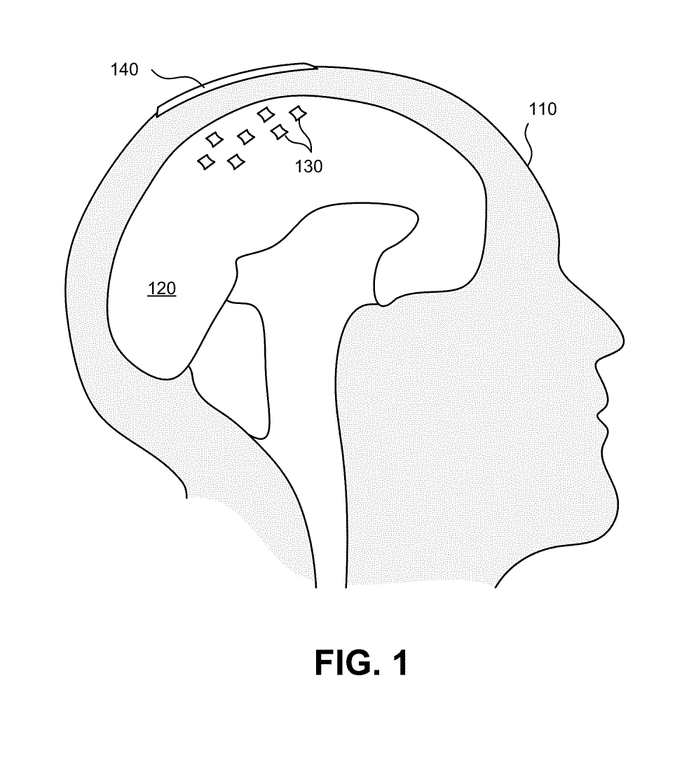

FIG. 1 is a simplified cross-sectional diagram illustrating an embodiment of a wireless medical implant system. Here, a patient's head 110 is illustrated, indicating a portion of the brain 120 in which a plurality of medical implants 130 are implanted. (For clarity, only a portion of the medical implants are labeled.) An interrogator device 140 uses low-power, short-range radio frequency (RF) signals at a designated frequency not only to communicate with the one or more medical implants, but also, in some embodiments, to provide power to the implants. Such wireless communication can employ any of a variety of short-range wireless technologies, including near-field communication (NFC) and/or other wireless technologies. According to some embodiments, data may be communicated in a secure fashion (e.g., using any of a variety of encryption techniques).

For scenarios in which the wireless medical implant system is utilized to measure and stimulate a portion of the brain (as shown in FIG. 1), the interrogator device 140 may be referred to as a “skin patch” because it may be substantially flat in shape and may be disposed on or near the patient's skin. The medical implants 130 in such scenarios may be referred to as “neurograins” because of their relatively small size and location within the patient's brain. A person of ordinary skill in the art will appreciate that alternative embodiments may be located in one or more other areas (other than the brain) of a patient's body and/or non-medical wireless communication applications.

Depending on the application, the wireless medical implant system may comprise hundreds or thousands of medical implants 130. (Alternative embodiments may include a smaller or larger number of medical implants 130 than this.) These medical implants 130 can also communicate back to the interrogator device 140 (e.g., through RF backscatter, by changing the impedance of their respective antennas) using, for example, a time division multiple access (TDMA) protocol. The interrogator device 140 may coordinate the uplink transmission.

Medical implants 130 can comprise active devices (having a power source) and/or passive devices (having no power source) configured to take biological measurements of the brain 120 (e.g., information regarding electrical signals generated by the patient's brain cells) and communicate the measurements to the interrogator device 140 and/or provide stimulation of the patient's brain 120 (e.g., via one or more electrodes), where such stimulation may be based on communication received from the interrogator device 140. As previously noted, medical implants 130 can be powered by the interrogator device 140 using, for example, a coiled antenna drawing power from communications and/or other signals or fields generated by the interrogator device 140. It can be noted that, in alternative embodiments, the interrogator device 140 may comprise multiple antennas, and/or the biological measurement and stimulation system may have one or more nodes and/or devices between the medical implants 130 and the interrogator device 140. Because medical implants 130 can vary in functionality, they can vary in size, shape, type, and/or may have electrodes (and or other sensors) that vary as well.

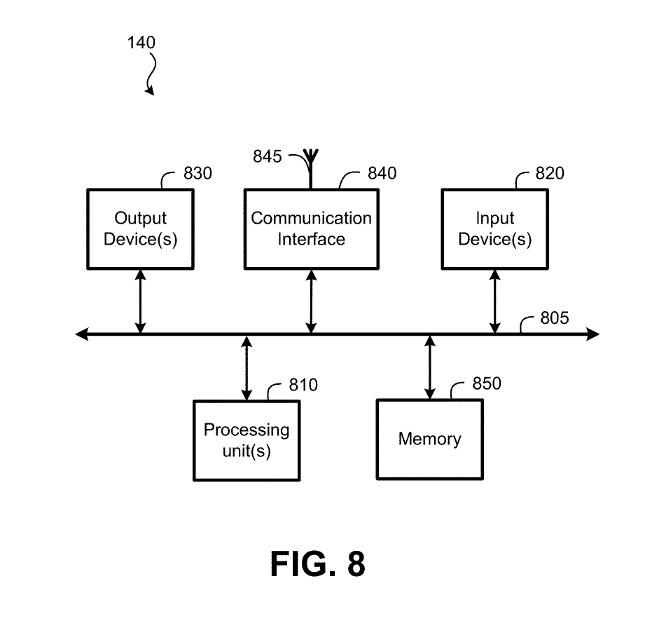

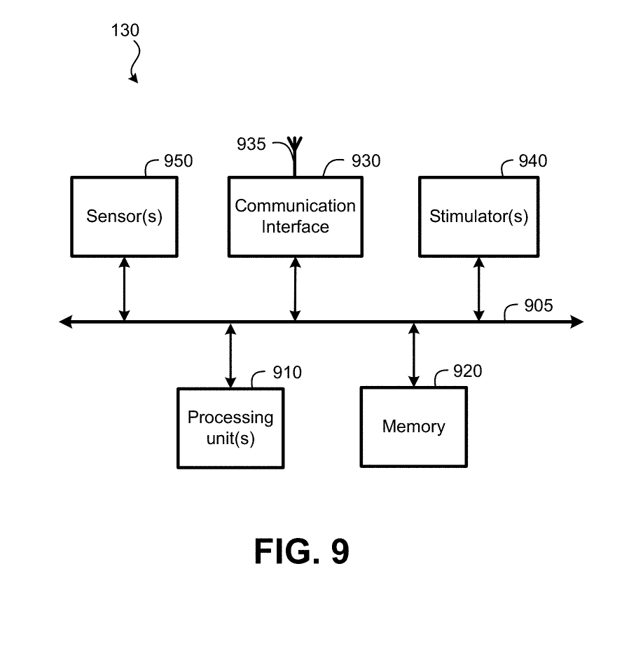

A person of ordinary skill in the art will appreciate the basic hardware configuration of an interrogator device 140 and/or medical implant 130. This can include, for example, a power source, processing unit, communication bus, volatile and/or non-volatile memory (which may comprise a non-transitory computer-readable medium having computer code for execution by the processing unit), transceiver, antenna, etc. The medical implant 130 may further comprise one or more sensors, electrodes, and/or stimulators utilized for sensing and/or stimulating one or more parts of the body. As such, the interrogator device 140 and/or medical implant 130 may have means for performing some, or all, of the functions described herein using one or more of its hardware and/or software components. In some embodiments, components may be selected and/or optimized for low power consumption. In particular, because medical implants 130 may be limited in size and/or power, the medical implants 130 may not have the same memory size and/or processing capabilities as the interrogator device 140. Example electrical hardware and software components of an interrogator device 140 and medical implant 130 are illustrated in FIG. 8 and FIG. 9, respectively, and described in more detail below.

As noted above, the medical implants may be passive or active implants that collect energy from an interrogator device, and thereby may need to operate on very low power consumption. But this can provide severe power constraints on the design of the local oscillator (LO) used for RF communication, because the accuracy of the LO is directly related to power consumption: low-power LOs are generally less accurate than relatively higher-power LOs. In addition, due to size constraints there is insufficient area to include a crystal on the medical device, making it more difficult to produce an accurate LO frequency. Thus, to preserve power in a medical implant system, wireless communication may need to tolerate relatively large inaccuracies in the carrier frequency. (Because an interrogator device is typically given a larger power budget than the medical implant, it may have a more accurate LO then the medical implant. The medical implant, however, may have a highly inaccurate LO.)

For example, in some embodiments, the LO of the medical implants (which may comprise a simple ring oscillator) can vary in frequency accuracy from approximately ±10% to approximately ±30%. Other embodiments may experience larger or smaller frequency inaccuracies. However, downlink communications from the interrogator device to the medical implants occurs at a low data rate, using, for example, only three bits per medical implant per frame.

Techniques disclosed herein are directed toward the providing a robust PWM amplitude modulation scheme that enables accurate synchronization and wireless data transmission in a wireless medical implant system despite large inaccuracies in the carrier frequency of such transmission, using a single RF carrier. In particular, the techniques provided herein enable medical implants with highly inaccurate LOs to reliably receive downlink communications from an interrogator device by using PWM amplitude modulation to enable synchronization and encode data.

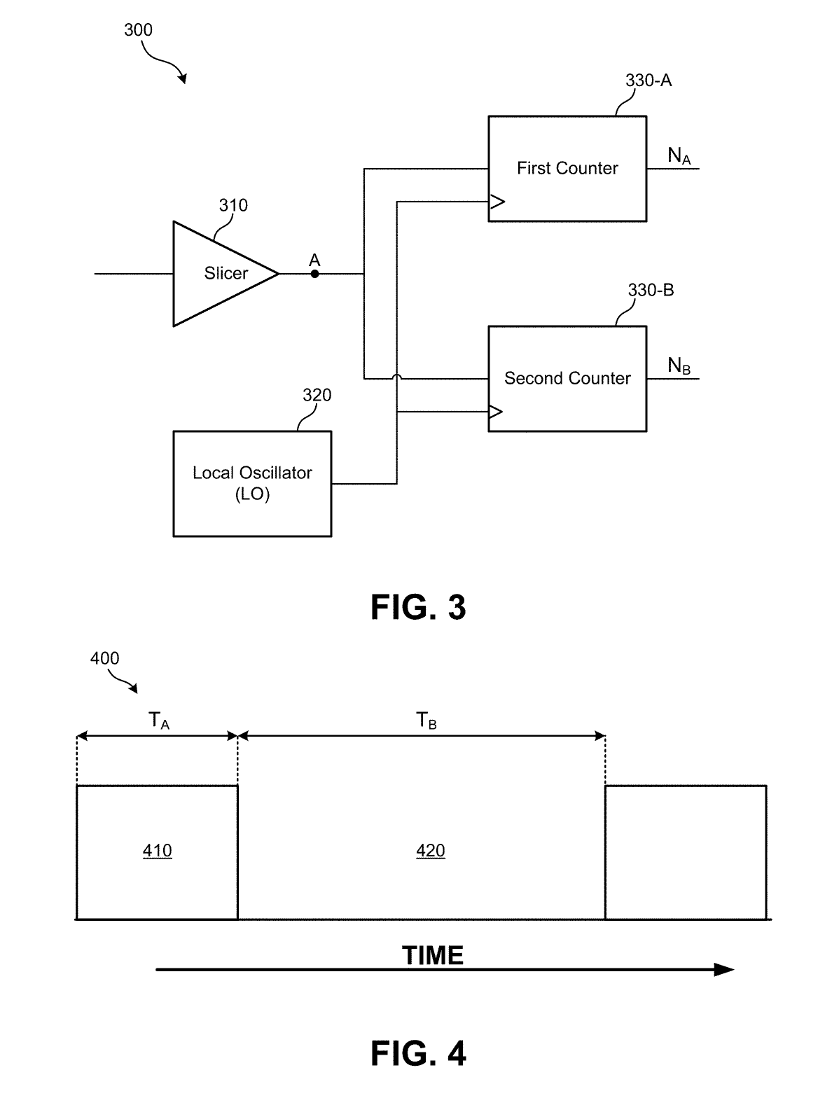

According to some embodiments, the PWM amplitude modulation can take advantage of the high Signal-to-Noise Ratio (SNR) the wireless medical implant system. As previously indicated, the interrogator device can provide power to the medical implant, which can result in a signal with a high SNR. This can enable a simple slicer (e.g., a comparator) to detect high and low amplitude modulation. (An example circuit is provided in FIG. 3 and described in more detail below.)

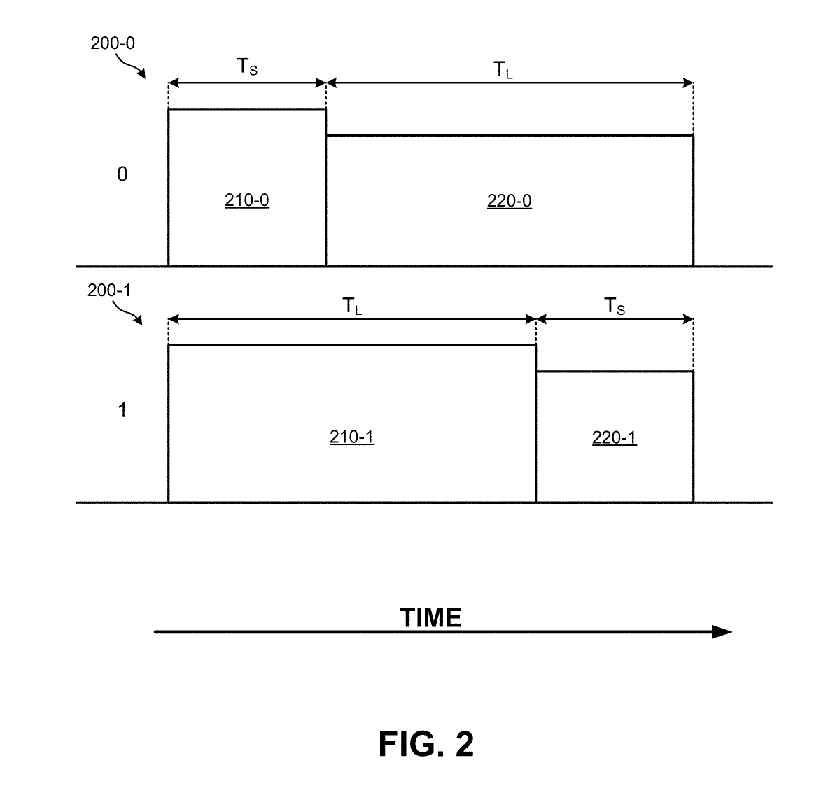

FIG. 2 is an illustration of how data can be encoded as pulse pairs 200 in the PWM amplitude modulation scheme, according to embodiments. Here, pulse pairs 200 are illustrated as pulses of amplitude (e.g., RF amplitude) over time, each pair 200 representing a different binary value. The first pulse pair 200-0 represents a digital “0”, and a second pulse pair 200-1 represents a digital “1”. Depending on the desired functionality, pulse pairs may comprise a first pulse 210 of “high” amplitude and a second pulse 220 of “low” amplitude. (This convention, however, is chosen arbitrarily. Alternative embodiments may choose to have a “low” first pulse and a “high” second pulse. Additionally, the amplitude of the “high” and “low” pulses may vary, depending on desired functionality, as long as a slicer of the medical implant is sufficiently able to reliably distinguish between these different amplitudes, thereby being able to distinguish the pulses within the pair of pulses by their amplitude.)

While the differing amplitudes of the pulses enable the medical implant to distinguish the first and second pulses within the pair of pulses, the information is encoded based on the relative pulse lengths. In particular, one of the pulses will have a short pulse duration of T_{S}, and the other pulse will have a long pulse of duration T_{L}. In the first pulse pair 200-0, the first (“high”) pulse 210-0 has a short pulse duration and the second (“low”) pulse 220-0 has a long pulse duration. In contrast, the second pulse pair 200-1, the first (“high”) pulse 210-1 has a long pulse duration and the second (“low”) pulse 220-1 has a long pulse duration. Thus, by identifying pulse pairs (a “high” pulse followed by a “low” pulse) and determining the relative length of pulses in each pair, a medical implant receiving these pulses (e.g., in a communication from the interrogator device) can decode the information encoded therein.

Ratio R can be computed from T_{L} and T_{S} as:

$$R = \frac{{T}_{L}}{{T}_{S}} \qquad ( 1 )$$

R can be a design parameter that can be set based on the inaccuracy of the LO of the medical implant. (The lower the accuracy, the larger R may be, to help the medical implant distinguish between long and short pulses. A person of ordinary skill in the art will appreciate the factors used to determine the value of R.) As indicated in FIG. 2, a typical value of R might be 2, but embodiments may have a smaller or larger values of R, depending on the accuracy of the LO of the medical implant.

As previously noted, because the SNR of the downlink signal may be relatively high, the difference between the amplitude of the “high” pulse and the “low” pulse may be relatively small. In FIG. 2, for example, the amplitude of the “low” pulse may be approximately 90% of the amplitude of the “high” pulse, resulting in an average power of 95% of the amplitude of the “high” pulse (in cases where the number of high and low pulses are approximately the same). This can be particularly helpful in systems where downlink pulses provide power to medical implants (or other passive devices). The difference between amplitudes of “high” and “low” pulses can vary, depending on desired functionality, capabilities of the medical implant, desired amount of power to transfer to medical implant, and/or other factors.

FIG. 3 is a schematic diagram of a circuit 300 that may be used by a medical implant to decode a signal encoded in the manner illustrated in FIG. 2. As previously mentioned, data received at the medical device may be decoded using a slicer 310 (or comparator) that distinguishes between high and low pulses. Again, because received power may be much higher than noise level, the SNR may be relatively high (e.g., −20 dB), resulting in a reliable distinction between high and low pulses received at the medical device from the interrogator device. The resulting output signal of the slicer 310 (e.g., at node A in FIG. 3) may be a binary signal.

An example binary output of the slicer 310 is illustrated in FIG. 4. Here, a first (high) pulse 410 of a pulse pair has a duration of T_{A} and a second (low) pulse 420 (illustrated with an amplitude of 0) of the pulse pair has a duration of T_{B}. These durations can then be compared (e.g., by calculating ratio R) to decode the bit encoded in the pulse pair as a logical one or a logical zero. Thus, inaccuracies in the LO 320 (referring again to FIG. 3) of a medical device decoding the pulse pair will have little or no effect on the accuracy of the decoding, because the ratio of the pulses is used for decoding, rather than a reliance on the accuracy of the LO 320. In some embodiments, Automatic Gain Control (AGC) may be used to set the threshold of the slicer 310 (e.g., at the midpoint between high and low amplitude modulation) to ensure accuracy in detecting high and low received signals. As indicated above, information is encoded in the pulse duration of amplitude modulated signals.

Again, the LO 320 at the medical implant may be designed for low power. One such LO 320 is a ring oscillator. Another such LO is an RC relaxation oscillator. Here, the frequency of the LO 320, f_{RO}, can be designed to be much higher than the inverse of the duration of the short pulse, to help ensure that measurements of the pulses are relatively accurate. That is:

$${f}_{RO} ≫ \frac{1}{{T}_{S}} \qquad ( 2 )$$

In some embodiments, for example, the f_{RO} may be 4 times (or more) than the inverse of the duration of the short pulse.

Counters 330 can then be used at the medical implant to measure the duration of the first pulse (T_{A}) and the duration of the second pulse (T_{B}). These counters can use the LO 320 of the medical implant as a reference clock, so they can count the number of clock transitions during the pulse. For instance, where is f_{RO} 10 times the inverse of the length of a short pulse, for example, the corresponding counter me count 10 clock transitions during the pulse.

In the embodiment illustrated in FIG. 3, a first counter 330-A and a second counter 330-B, are used to determine the respective lengths of the first (high) pulse and the second (low) pulse of a received bit. More specifically, when the slicer 310 detects a “high” input signal and outputs a corresponding logical “high” pulse, the first counter 330-A begins counting up to a number N_{A}. And when the slicer 310 detects a “low” input signal and outputs a corresponding logical “low” pulse, the first counter 330-A stops counting and the second counter 330-B begins counting up to a number N_{B}. (It will be understood that counters 330 may be designed to have enough bits, in view of the received pulses, the frequency of the LO 320, and the inaccuracy of the LO 320, so that they do not roll over while counting.)

Decoding the information may proceed as follows, according to some embodiments. After values of values N_{A} and N_{B} are determined (e.g., once the next “high” pulse is detected, signaling the beginning of the next pulse pair) the values N_{A} and N_{B} can then be compared, effectively comparing the relative lengths of the first and second pulses to determine the encoded bit. If N_{A} is less than or equal to N_{B} then the decode value is a zero, and if N_{A} is greater than N_{B} then the decode value is a one. (Of course, whether the decode value is a one or a zero when the values of N_{A} and N_{B} are equal is arbitrary, depending on desired functionality.) The counters can then be reset to decode the next bit. (As previously mentioned, a medical implant may receive three bits of information per frame, in an example embodiment. Other embodiments may include a larger or smaller number of bits per medical implant per frame.)

In this manner, the medical implant can decode a signal even when clock frequency is highly inaccurate. This is because information is encoded in the ratio of the width of the two pulses. Thus, even when the clock is off by 30% or even more, the decoding by the medical implant remains highly accurate. This is the case even when clock jitter leads to errors in the counter values, as long as the value of R (the ratio of the pulse widths) is sufficiently high. Again, the value of R may be set to accommodate large inaccuracies in the LO 320 of the medical implant.

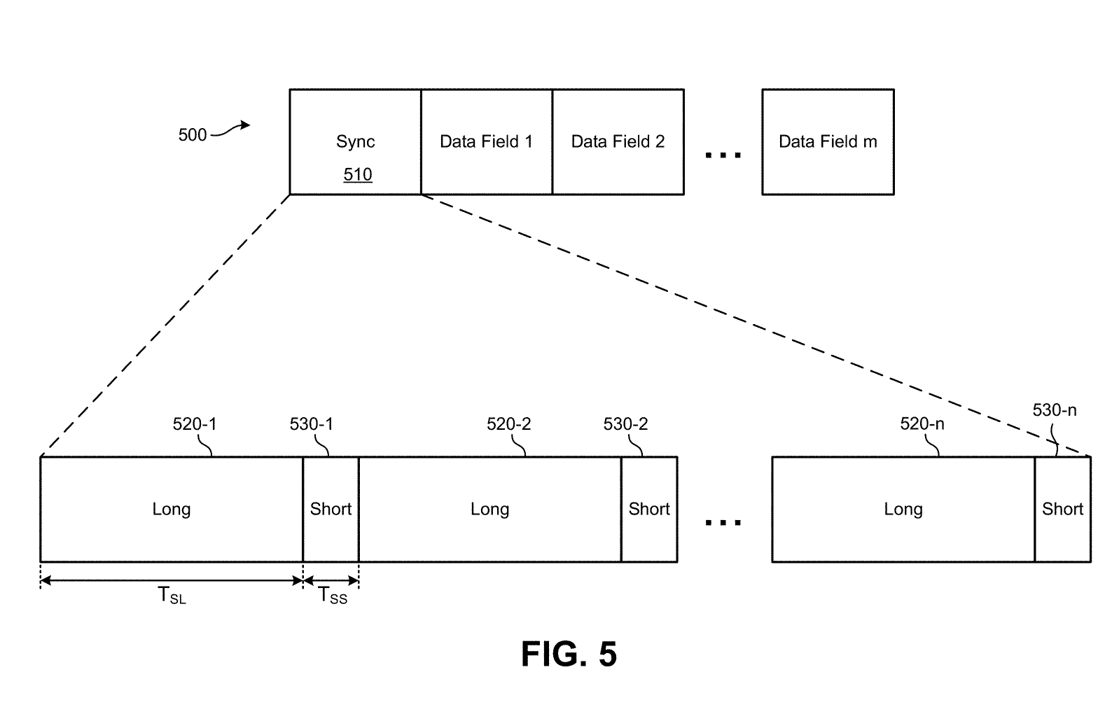

FIG. 5 is a diagram of a communication frame 500 received in the downlink signal that may be used for synchronization of the medical implants, according to embodiments. As previously indicated, the interrogator device may be in communication with many medical implants. Interrogator device may engage in downlink communication to the various medical implants using a communication frame 500 comprising a synchronization field 510 followed by data fields for the various medical implants. In the communication frame 500 illustrated in FIG. 5, there are m data fields, where the number m of data fields can vary depending on the number of medical implants and/or other factors.

The determination of which data field corresponds to which medical implant may vary, depending on desired functionality. In some embodiments, for example, the interrogator device may designate data fields to medical implants. In other embodiments, this may be predetermined such that an address of the medical implant corresponds to a data field in the frame. A person of ordinary skill in the art will appreciate that various ways in which such determinations may be made.

To help ensure that medical implants synchronize to the synchronization field 510 rather than the data fields, the pulse widths of the pulses in the synchronization field can be different to those in the data fields. For example, as illustrated, data in the synchronization field 510 may be encoded using pulse pairs of long and short pulses (a manner similar to the data encoding as illustrated in FIG. 2). (Although not shown in FIG. 5, pulse pairs may comprise pairs of pulses having different amplitudes, in the manner similar to the pulse pairs illustrated in FIG. 2, such as a “high” pulse followed by an “low” pulse.) In FIG. 5, the example synchronization field comprises a series of n pulse pairs, each comprising a long pulse (520-1, 520-2, . . . , 520-n, collectively and generically referred to herein as long pulses 520) followed by a short pulse (530-1, 530-2, . . . , 530-n, collectively and generically referred to herein as short pulses 520). (It will be understood that the synchronization pattern of long pulses 520 followed by short pulses 530 is provided in FIG. 5 as an example. Other synchronization patterns may be utilized, depending on desired functionality. Additionally, it will be understood that although “n” pairs of synchronization pulses are illustrated, any number of pulse pairs may be used, including 1 or 2 pairs.)

To distinguish the pulse pairs in the synchronization field 510 with other encoded data, the ratio of the long pulses 520 to short pulses 530 can be different. According to some embodiments, for example, the ratio K of the duration (T_{SL}) of the long pulses 520 in the synchronization field 510 to the duration (T_{SS}) of the short pulses 530 in the synchronization field 510 is distinguishably different (from the perspective of the medical implants) than R (the ratio of the duration a long data pulse to a short data pulse). For example, in embodiments where R may equal 2, the ratio K may equal 6. (Because there is only one synchronization field in a frame, lengthening the pulses in the synchronization field does not result in much additional overhead of the frame.) In other words, the duration of the long pulse 520 of the synchronization field can be much larger than a long pulse in a data field, so that, even if an edge transition is missed, the medical implant can identify the long pulses of the synchronization field.

To determine the difference between the R and K (and thereby distinguish between data and synchronization fields) the medical device may compare the absolute ratio of the pulses in the sync field (rather than simply determine which pulse is longer, as it may do when decoding data in the data field). As such, corresponding circuitry may be used to make this comparison, which may also use counters. A person of ordinary skill in the art will appreciate that this may be implemented in various ways using various circuits.

Similar to the circuit illustrated in FIG. 3, two counters can be used to count the duration of the pulses using a circuit. (In some embodiments, the use two counters may be in addition to the two counters in the circuit 300 in FIG. 3, thereby making four counters.) A first counter can be used to count the duration of the “high” pulse, while a second counter can be used to count the duration of the “low” pulse. If only a single “high long pulse” and a single “low short pulse” are used, then after counting the length of the first (high) pulse with the first counter and the second (low) pulse with the second counter, the synchronization field 510 is detected if the ratio of the first counter output to the second counter output is significantly high (e.g. greater than 4). If the ratio is does not exceed the threshold, then a synchronization field 510 has not been detected. The circuit can then continue to search for the synchronization field 510 by checking the next pair of “high” and “low” pulses.

Each individual medical implant can track synchronization to identify its corresponding data field in the data frame. Because the accuracy of the LO of the medical implant is poor, the medical implant may not be able to simply rely on the LO for timing. However, using the techniques provided herein above, the medical implant can accurately detect the various bits transmitted in the data frame. Thus, the medical implant can locate its data field in the data frame by keeping track of the bits of each field in the data frame to determine when its corresponding data field arrives (because each data field can have a predetermined length). For instance, if the predetermined length of each data field is 3 bits, a medical implant having the data field 5 can identify data field 5 by tracking (counting) the bits of the first 4 data fields (3 bits*4 data fields=12 bits (or 24 pulse pairs)) and further identify the 3 bits that follow as the 3 bits of data field 5.

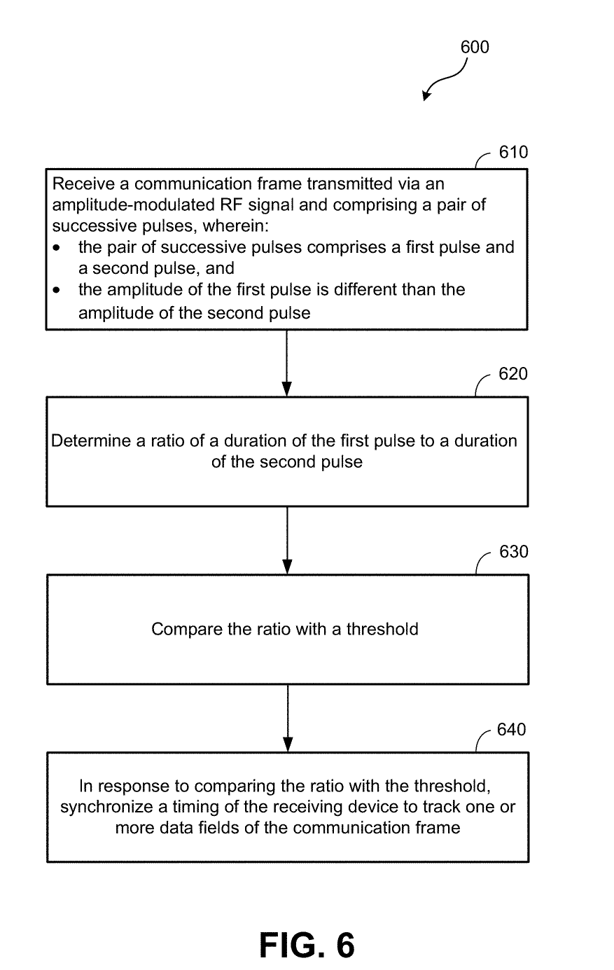

FIG. 6 is a flow diagram of an example method 600 of decoding wireless communication at a receiving device, according to another embodiment. Here, the functionality described in one or more of the blocks illustrated in FIG. 6 may be performed, for example, by a receiving device in a low-power wireless system, such as a medical implant 130 of the wireless medical implant system illustrated in FIG. 1. Accordingly, means for performing this functionality may include hardware and/or software components of a medical implant 130. An example of such hardware and/or software components is illustrated in FIG. 9 and described in more detail below.

The functionality at block 610 includes receiving a communication frame transmitted via an amplitude-modulated RF signal and comprising a pair of successive pulses, where the pair of successive pulses comprises a first pulse and a second pulse, and the amplitude of the first pulse is different than the amplitude of the second pulse. The RF signal, in some embodiments, may power the receiving device, at least in part. Moreover, as illustrated in the embodiments above, first and second pulses may be identified by their respective amplitudes (e.g., each pair starting with a higher-amplitude pulse, or vice versa, depending on convention). Moreover, in some embodiments, a comparator (slicer) may be used to distinguish the amplitude of the first pulse from the amplitude of the second pulse. Here, the communication frame may comprise a synchronization field and a plurality of data fields, similar to the synchronization frame 500 illustrated in FIG. 5. As further illustrated in FIG. 5, the synchronization field 510 may comprise one or more pulse pairs, each pair having a long pulse and a short pulse (the long pulse being relatively longer induration than the short pulse). Means for performing the functionality at block 610 may comprise, for example, bus 905, processing unit(s) 910, memory 920, communication interface 930, antenna 935, and/or other components of a medical implant 130, as illustrated in FIG. 9 and described in more detail below.

At block 620, the functionality comprises determining a ratio of a duration of the first pulse to a duration of the second pulse. As indicated in the embodiments above, this can be done, for example, by using a first counter to determine the duration of the first pulse and using a second counter to determine the duration of the second pulse. (Of course, it will be understood that determining a ratio of the duration of the second pulse to the duration of the first pulse is implicit in determining the ratio of the duration of the first pulse to the duration of the second pulse. The actual ratio computed is a matter of convention.) Means for performing the functionality at block 620 may comprise, for example, bus 905, processing unit(s) 910, memory 920, and/or other components of a medical implant 130, as illustrated in FIG. 9 and described in more detail below.

At block 630, the ratio is compared with a threshold. As noted above, the ratio of long to short pulses in synchronization field may be distinguishably larger than the ratio of long to short pulses in data fields. As such, the ratio can be set to distinguish between pulses in data fields and pulses in the synchronization field. Thus, if a ratio meets or exceeds (this can be, for example, either a maximum threshold, or minimum threshold, depending on whether the ratio is long to short or short to long), then the pulses can be identified as pulses in a synchronization field. As example, if the long to short ratio meets or exceeds 4:1, then it can be determined that the pulses comprise a pulse pair in a synchronization field. Means for performing the functionality at block 630 may comprise, for example, bus 905, processing unit(s) 910, memory 920, and/or other components of a medical implant 130, as illustrated in FIG. 9 and described in more detail below.

The functionality at block 640 comprises, in response to comparing the ratio with the threshold, synchronizing a timing of the receiving device to track one or more data fields of the communication frame. As noted in the embodiments above, identifying a synchronization field of a communication frame can enable a medical implant to track data fields in the communication frame (and thereby identify the data field corresponding to and intended for the medical implant). The medical implant can track the data frames by, for example, identifying pulse pairs of a data frame (e.g., by comparing the duration of each pulse in a pulse pair) and simply counting the pulse pairs (e.g., digital bits). Because the number of bits may be pre-determined for each data frame, the number of data frames can be tracked by tracking the number of pulse pairs. Means for performing the functionality at block 640 may comprise, for example, bus 905, processing unit(s) 910, memory 920, and/or other components of a medical implant 130, as illustrated in FIG. 9 and described in more detail below.

Some embodiments may include additional functionality. For instance, in some embodiments, the method 600 may further comprise receiving a second pair of success of pulses in a data field of the communication frame, where the second pair of successive pulses comprise a third pulse and a fourth pulse. Again, the amplitude of the third pulse may be different than the amplitude of the fourth pulse (enabling identification of pulse pairs). A duration of the third pulse can be compared with a duration of the fourth pulse, and a digital bit of information encoded by the third pulse and the fourth pulse can be determined, based on the comparison of the duration of the third pulse with the duration of the fourth pulse. In some embodiments, the receiving device can determine, from the communication frame, a data field corresponding to the receiving device by counting a number of bits in the communication frame. As previously indicated, these bits can include, for example, bits of data in data fields.

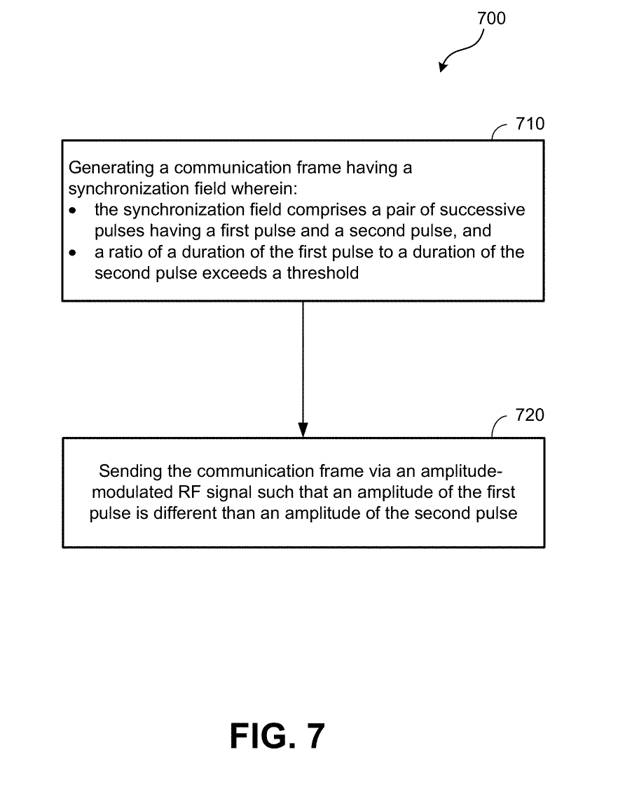

FIG. 7 is a flow diagram of an example method 700 of encoding wireless information at a transmitting device, according to an embodiment. The functionality described in one or both blocks illustrated in FIG. 7 may be performed, for example, by a transmitting device in a low-power wireless system, such as an interrogator device 140 of the wireless medical implant system illustrated in FIG. 1. Accordingly, means for performing this functionality may include hardware and/or software components of an interrogator device 140. An example of such hardware and/or software components is illustrated in FIG. 8 and described in more detail below.

The functionality at block 710 comprises generating a communication frame having a synchronization field, where the synchronization field comprises a pair of successive pulses having a first pulse and a second pulse, and a ratio of the duration of the first pulse to a duration of the second pulse exceeds a threshold. Here again, pulse pairs in a synchronization field may have a long-to-short ratio longer than a similar ratio of pulse pairs in a data field. To enable a receiving device to make the distinction, the transmitting device can ensure that the ratio exceeds a threshold at which the receiving device can make the distinction. Means for performing the functionality at block 710 may comprise, for example, bus 805, processing unit(s) 810, memory 850, and/or other components of an interrogator device 140, as illustrated in FIG. 8 and described in more detail below.

The functionality at block 720 comprises sending the communication frame via an amplitude-modulated RF signal such that an amplitude of the first pulse is different than an amplitude of the second pulse. As noted above, amplitudes may differ (e.g., one “high” pulse and one “low” pulse) to signify pulse pairs, and the amplitudes themselves may be chosen based on desired functionality, power transfer, convention, etc. Means for performing the functionality at block 720 may comprise, for example, bus 805, processing unit(s) 810, memory 850, communication interface 840, antenna 845, and/or other components of an interrogator device 140, as illustrated in FIG. 8 and described in more detail below.

Of course, the communication frame may include data fields as well, which may be encoded as described herein above (e.g., as shown in FIG. 2). Therefore, in some embodiments, the method may further comprise encoding, in the communication frame, a digital bit of information in a third pulse and a fourth pulse, where the value of the digital bit is based on a ratio of the duration of the third pulse compared to a duration of the fourth pulse. Moreover, to help ensure the pulse pair of the data frame is not confused with a pulse pair of a synchronization frame, the ratio duration of the third pulse compared to the duration of the fourth pulse may not exceed the threshold.

FIG. 8 is a simplified block diagram of an interrogator device 140, according to an embodiment. The interrogator device 140 may comprise a “skin patch” (similar to the interrogator device of FIG. 1) or other device configured to perform one or more of the functions of an interrogator device as described in embodiments herein. FIG. 8 is meant only to provide a generalized illustration of various components, any or all of which may be included or omitted as appropriate. The interrogator device 140 may be configured to execute one or more functions of the methods described herein, such as the method 700 illustrated in FIG. 7. It can be further noted that the interrogator device 140 may be configured to receive measurements from and/or stimulate a body part utilizing one or more medical implants with which the interrogator device 140 is in wireless communication, as described in the embodiments above. In some embodiments, the particular measurements taken and/or stimulations may be determined by the interrogator device 140 itself, and/or be determined by another device (such as a medical device, mobile phone, tablet, etc.) with which the interrogator device 140 is in communication. A person of ordinary skill in the art will understand that, for the sake of simplicity, some components (e.g., power source, clock, physical housing, etc.) are not shown.

The interrogator device 140 is shown comprising hardware elements that can be electrically coupled via a bus 805 (or may otherwise be in communication, as appropriate). The hardware elements may include a processing unit(s) 810 which may comprise without limitation one or more general-purpose processors, one or more special-purpose processors (such as digital signal processing (DSP) chips, graphics acceleration processors, application specific integrated circuits (ASICs), and/or the like), and/or other logic, processing structure, or means, which can be configured to perform one or more of the methods described herein.

Depending on desired functionality, the interrogator device 140 also may comprise one or more input devices 820, which may comprise without limitation one or more, touch sensors, buttons, switches, and/or more sophisticated input components, which may provide for user input, which may enable the system to power on, configure operation settings, and/or the like. Output device(s) 830 may comprise, without limitation, light emitting diode (LED)s, speakers, and/or more sophisticated output components, which may enable feedback to a user, such as an indication the implant system has been powered on, is in a particular state, is running low on power, and/or the like.

The interrogator device 140 might also include a communication interface 840 and one or more antennas 845. This communication interface 840 and antenna(s) 845 can enable the interrogator device 140 to communicate with and optionally power the medical implants of the wireless medical implant system. The one or more antennas 845 can be configured to, when powered properly, generate particular signals and/or fields to communicate with and/or power the medical implants, including communicating medical implant selection methods as described herein. As previously indicated, medical implants in some embodiments may communicate using RF backscatter, in which case the interrogator device 140 may transmit an RF carrier signal, modulated by the medical implants during uplink communications.

In some embodiments, the processing unit(s) 810 and/or communication interface 840 (including software and/or firmware executed therewith) may create a communication frame and/or perform amplitude modulation of an RF signal to encode the RF signal with the communication frame, as described herein.

The communication interface 840 may further enable the interrogator device 140 to communicate with one or more devices outside the biological measurement and stimulation system to which the interrogator device 140 belongs, such as a medical device, mobile phone, tablet, etc. In some embodiments, the one or more devices may execute a software application that provides a user interface (e.g., a graphical user interface) for configuring and/or managing the operation of the interrogator device 140. The communication interface may include connectors and/or other components for wired communications (e.g., universal serial bus (USB) Ethernet, optical, and/or other communication). Additionally, or alternatively, the communication interface 840 and optionally the antenna(s) 845 may be configured to provide wireless communications (e.g., via Bluetooth®, Bluetooth® low energy (BLE), Institute of Electrical and Electronics Engineers (IEEE) 802.11, IEEE 802.15.4 (or ZIGBEE®), Wi-Fi, WiMAX™, cellular communications, infrared, etc.). As such, the communication interface 840 may comprise without limitation a modem, a network card, an infrared communication device, a wireless communication device, and/or a chipset.

The interrogator device 140 may further include and/or be in communication with a memory 850. The memory 850 may comprise, without limitation, local and/or network accessible storage such as optical, magnetic, solid-state storage (e.g., random access memory (“RAM”) and/or a read-only memory (“ROM”)), or any other non-transitory, computer-readable medium. The memory 850 may therefore make the interrogator device 140 can be programmable, flash-updateable, and/or the like. Such storage devices may be configured to implement any appropriate data stores, including without limitation, various file systems, database structures, and/or the like.

The memory 850 of the interrogator device 140 also can comprise software elements (not shown), including an operating system, device drivers, executable libraries, and/or other code, such as one or more application programs, which may comprise computer programs provided by various embodiments, and/or may be designed to implement methods, and/or configure systems, provided by other embodiments, as described herein. For example, one or more procedures described with respect to the functionality discussed above might be implemented as computer code and/or instructions executable by the interrogator device 140 (and/or processing unit(s) 810 of the interrogator device 140). The memory 850 may therefore comprise non-transitory machine-readable media having the instructions and/or computer code embedded therein/thereon.

FIG. 9 is a simplified block diagram of a medical implant 130, according to an embodiment. The medical implant 130 may comprise a “neurograin” (similar to the medical implants 130 of FIG. 1) or other device configured to perform one or more of the functions of a medical implant of a biological measurement and stimulation system as described in embodiments herein. FIG. 9 is meant only to provide a generalized illustration of various components, any or all of which may be included or omitted as appropriate. It can be further noted that the medical implant 130 may be configured to take measurements and/or stimulate a body part as directed by an interrogator device 140 using communications such as those described in the embodiments herein. A person of ordinary skill in the art will understand that, for the sake of simplicity, some components (e.g., power source, LO, physical housing, etc.) are not shown. It will be understood that, in most embodiments, hardware and/or software optimizations may be made to help minimize power consumption.

The medical implant 130 is shown comprising hardware elements that can be electrically coupled via a bus 905, or may otherwise be in communication, as appropriate. The hardware elements may include a processing unit(s) 910 which may comprise without limitation one or more general-purpose processors, one or more special-purpose processors (e.g., microprocessors), and/or other logic, processing structure, or means, which can be configured to perform one or more of the methods described herein. As a person of ordinary skill in the art will appreciate, the processing unit(s) 910, may further include one or more splicers, counters, and/or other circuitry as described herein (and/or may implement the functions of such circuitry and software) for processing incoming RF signal. Additionally or alternatively, such circuitry (or software) he be implemented in the communication interface 930, described in more detail below.

The medical implant 130 may further include and/or be in communication with a memory 920. As with other components of the medical implant 130, the memory 920 may be optimized for minimum power consumption. In some embodiments, the memory 920 may be incorporated into the processing unit(s) 910. Depending on desired functionality, the memory (which can include a non-transitory computer-readable medium, such as a magnetic, optical, or solid-state medium) may include computer code and/or instructions executable by the processing unit(s) 910 to perform one or more functions described in the embodiments herein.

A communication interface 930 and antenna(s) 935 can enable the medical implant 130 to wirelessly communicate the interrogator device, as described herein. The antenna(s) 935 may comprise a coiled or other antenna configured to draw power from communications and/or other signals or fields generated by the interrogator device, powering the medical implant 130. In some embodiments, the medical implant 130 may further include an energy storage medium (e.g., a battery, capacitor, etc.) to store energy captured by the antenna(s) 935. In some embodiments, the communication interface 930 and antenna(s) 935 may be configured to the interrogator device using RF backscatter, as noted above.

The stimulator(s) 930 of the medical implant 130 can enable the medical implant 130 to provide stimulation to a body part (e.g., biological tissue) in which the medical implant 130 is implanted. As such, the stimulator(s) 940 may comprise an electrode, LED, and/or other component configured to provide electrical, optical, and/or other stimulation. The processing unit(s) 910 may control the operation of the stimulator(s) 940, and may therefore control the timing, amplitude, and/or other stimulation provided by the stimulator(s) 940.

The sensor(s) 950 may comprise one or more sensors configured to receive input from a body part (e.g., biological tissue), in which the medical implant 130 is implanted. Sensors may therefore be configured to sense electrical impulses, pressure, temperature, light, conductivity/resistivity, and/or other aspects of a body part. As described herein, embodiments may enable medical implant 130 to provide this information, via the communication interface 930, to an interrogator. Depending on desired functionality, information received by the sensor(s) 950 may be encrypted, compressed, and/or otherwise processed before it is transmitted via the communication interface 930.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus, many of the elements are examples that do not limit the scope of the disclosure to those specific examples.

It has proven convenient at times, principally for reasons of common usage, to refer to such signals as bits, information, values, elements, symbols, characters, variables, terms, numbers, numerals, or the like. It should be understood, however, that all of these or similar terms are to be associated with appropriate physical quantities and are merely convenient labels. Unless specifically stated otherwise, as is apparent from the discussion above, it is appreciated that throughout this Specification discussions utilizing terms such as “processing,” “computing,” “calculating,” “determining,” “ascertaining,” “identifying,” “associating,” “measuring,” “performing,” or the like refer to actions or processes of a specific apparatus, such as a special purpose computer or a similar special purpose electronic computing device. In the context of this Specification, therefore, a special purpose computer or a similar special purpose electronic computing device is capable of manipulating or transforming signals, typically represented as physical electronic, electrical, or magnetic quantities within memories, registers, or other information storage devices, transmission devices, or display devices of the special purpose computer or similar special purpose electronic computing device.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AA, AAB, AABBCCC, etc.

Having described several embodiments, various modifications, alternative constructions, and equivalents may be used without departing from the spirit of the disclosure. For example, the above elements may merely be a component of a larger system, wherein other rules may take precedence over or otherwise modify the application of the invention. Also, a number of steps may be undertaken before, during, or after the above elements are considered. Accordingly, the above description does not limit the scope of the disclosure.

What is claimed is:

1. A method of decoding wireless information at a receiving device, the method comprising:
receiving, at the receiving device, a communication frame transmitted via an amplitude-modulated radio frequency (RF) signal and comprising a pair of successive pulses, wherein:
the pair of successive pulses comprises a first pulse and a second pulse, and
an amplitude of the first pulse is different than an amplitude of the second pulse;
determining a ratio of a duration of the first pulse to a duration of the second pulse;
comparing the ratio with a threshold; and
in response to comparing the ratio with the threshold, synchronizing a timing of the receiving device to track one or more data fields of the communication frame.

2. The method of claim 1, further comprising:
using a first counter to determine the duration of the first pulse, and
using a second counter to determine the duration of the second pulse.

3. The method of claim 1, further comprising using a comparator to distinguish the amplitude of the first pulse from the amplitude of the second pulse.

4. The method of claim 1, further comprising:
receiving, in a data field of the communication frame, a second pair of successive pulses, wherein:
the second pair of successive pulses comprises a third pulse and a fourth pulse, and
the amplitude of the third pulse is different than the amplitude of the fourth pulse;
comparing a duration of the third pulse with a duration of the fourth pulse; and
determining a digital bit of information encoded by the third pulse and the fourth pulse based on the comparison of the duration of the third pulse with a duration of the fourth pulse.

5. The method of claim 4, further comprising determining, from the communication frame, a data field corresponding to the receiving device by counting a number of bits in the communication frame.

6. The method of claim 1, further comprising powering the receiving device, at least in part, using energy from the amplitude-modulated RF signal.

7. The method of claim 6, wherein the receiving device comprises a medical implant.

8. The method of claim 7, wherein the medical implant is implanted in a patient's brain.

9. A method of encoding wireless information at a transmitting device, the method comprising:
generating, at a transmitting device, a communication frame having a synchronization field wherein:
the synchronization field comprises a pair of successive pulses having a first pulse and a second pulse, and
a ratio of a duration of the first pulse to a duration of the second pulse exceeds a threshold; and
sending, with the transmitting device, the communication frame via an amplitude-modulated radio frequency (RF) signal such that an amplitude of the first pulse is different than an amplitude of the second pulse.

10. The method of claim 9, further comprising:
encoding, in the communication frame, a digital bit of information in a third pulse and a fourth pulse, wherein a value of the digital bit is based on a ratio of a duration of the third pulse compared to a duration of the fourth pulse, and wherein the ratio of the duration of the third pulse compared to a duration of the fourth pulse does not exceed the threshold.

11. The method of claim 9, wherein the transmitting device comprises an interrogator device configured to communicate with a medical implant implanted in a patient's brain.

12. A medical device comprising:
a wireless communication interface configured to receive a communication frame transmitted via an amplitude-modulated radio frequency (RF) signal comprising a pair of successive pulses wherein:
the pair of successive pulses comprises a first pulse and a second pulse, and
an amplitude of the first pulse is different than an amplitude of the second pulse;
a memory; and
a processing unit communicatively coupled with the wireless communication interface and the memory and configured to:
determine a ratio of a duration of the first pulse to a duration of the second pulse;
compare the ratio with a threshold; and
synchronize a timing of the medical device, based on the comparison of the ratio with the threshold, to track one or more data fields of the communication frame.

13. The medical device of claim 12, further comprising:
a first counter configured to determine the duration of the first pulse, and
a second counter configured to determine the duration of the second pulse.

14. The medical device of claim 13, wherein the first counter and the second counter compose part of the wireless communication interface or the processing unit.

15. The medical device of claim 12, further comprising a comparator configured to distinguish the amplitude of the first pulse from the amplitude of the second pulse.

16. The medical device of claim 15, wherein the comparator composes part of the wireless communication interface or the processing unit.

17. The medical device of claim 12, wherein:
the wireless communication interface is further configured to:
receive, in a data field of the communication frame, a second pair of successive pulses, wherein:
the second pair of successive pulses comprises a third pulse and a fourth pulse, and
the amplitude of the third pulse is different than the amplitude of the fourth pulse; and
the processing unit is further configured to:
compare a duration of the third pulse with a duration of the fourth pulse; and
determine a digital bit of information encoded by the third pulse and the fourth pulse based on the comparison of the duration of the third pulse with a duration of the fourth pulse.

18. The medical device of claim 17, wherein the wireless communication interface or the processing unit is further configured to determine, from the communication frame, a data field corresponding to the medical device by counting a number of bits in the communication frame.

19. The medical device of claim 18, wherein the wireless communication interface or the processing unit is configured to count the number of bits in the communication frame using a counter.

20. The medical device of claim 12, wherein the medical device is further configured to be powered, at least in part, using energy from the amplitude-modulated RF signal.

21. The medical device of claim 12, wherein the medical device comprises a medical implant.

22. The medical device of claim 21, wherein the medical implant is configured to be implanted in a patient's brain.

23. An interrogator device comprising:
a memory;
a processing unit communicatively coupled with the memory and configured to:
generate a communication frame having a synchronization field wherein:
the synchronization field comprises a pair of successive pulses having a first pulse and a second pulse, and
a ratio of a duration of the first pulse to a duration of the second pulse exceeds a threshold; and
a wireless communication interface communicatively coupled with the processing unit and configured to send the communication frame via an amplitude-modulated radio frequency (RF) signal such that an amplitude of the first pulse is different than an amplitude of the second pulse.

24. The interrogator device of claim 23, wherein the processing unit or wireless communication interface is further configured to encode, in the communication frame, a digital bit of information in a third pulse and a fourth pulse, wherein a value of the digital bit is based on a ratio of a duration of the third pulse compared to a duration of the fourth pulse, and wherein the ratio of the duration of the third pulse compared to a duration of the fourth pulse does not exceed the threshold.

25. The interrogator device of claim 23, wherein the interrogator device is configured to communicate with a medical implant implanted in a patient's brain.
